(19) United States
(12) Patent Application Publication
(10) Pub. No.: US 2024/0415862 A1
(43) Pub. Date: Dec. 19, 2024

(54) METHODS FOR TREATMENT OF MENOPAUSAL SYNDROMES

(71) Applicants: ELYSIUM HEALTH INC., New York, NY (US); UNIVERSITY OF SOUTHERN ALABAMA, Mobile, AL (US)

(72) Inventors: Ryan DELLINGER, New York, NY (US); Marie MIGAUD, Mobile, AL (US)

(21) Appl. No.: 18/703,429

(22) PCT Filed: Oct. 12, 2022
(86) PCT No.: PCT/US2022/077944

§ 371 Date: Apr. 22, 2024

Related U.S. Application Data
(60) Provisional application No. 63/272,217, filed on Oct. 27, 2021.

(51) Int. Cl. A61K 31/706; A61K 31/09; A61K 31/455; A61P 15/00

(52) U.S. Cl. CPC A61K 31/706; A61K 31/09; A61K 31/455; A61P 15/00

(57) ABSTRACT

The present application provides methods for reducing the severity or frequency of menopausal symptoms by administering (a) a therapeutically effective amount of nicotinamide riboside, nicotinamide mononucleotide, nicotinamide, nicotinic acid, the reduced form of nicotinamide riboside (NRH), or a mixture thereof, and (b) a therapeutically effective amount of pterostilbene. The compositions can be administered orally or via other suitable administration routes.

### FIELD OF THE INVENTION

The present application provides methods for reducing the severity or frequency of menopausal symptoms by administering (a) a therapeutically effective amount of nicotinamide riboside, nicotinamide mononucleotide, nicotinamide, nicotinic acid, the reduced form of nicotinamide riboside (NRH), or a mixture thereof, and (b) a therapeutically effective amount of pterostilbene. The compositions can be administered orally or via other suitable administration routes.

### BACKGROUND OF THE INVENTION

Menopause signals the end of the natural reproductive potential, coinciding with a decline in hormone levels for women. Typically, a well-orchestrated sequence of estrogen, progesterone, and testosterone production occurs each month with ovulation. The estrogen hormone estradiol plays a major role in reproduction.

As we age, sex hormone levels start to fluctuate and eventually decline significantly. Because brain, muscle, liver, skin and fat cells all have hormone receptors, this decline significantly and negatively affects skeletal, skin and nervous system functions, and is often associated with menopausal discomfort. This period of fluctuation and decline can last for upwards of 10 years prior to the last menstrual cycle (peri-menopause).

The climacteric syndrome is characterized by several symptoms: hot flashes are the most common and reported by about 70% of peri-post-menopausal women. Sleep disorders, particularly decreased sleep quality, mood swings, and irritability are also commonly reported.

Critically, estradiol levels steadily decrease over the course of peri and post menopause; a process that has been correlated to the occurrence and severity of menopausal symptoms. Therefore, estradiol is used in hormone replacement therapies for menopausal symptoms. However, excess estradiol is also associated with the development and progression of cancers such as breast, ovarian and endometrial cancer and therefore supplementation with estradiol is to be considered with care. Consequently, there is a strong need for a safer therapy for menopausal symptoms.

### BRIEF SUMMARY OF THE INVENTION

The present invention fulfills these needs by providing compositions and methods for treating a symptom of menopause in a female. These symptoms include hot flashes, decreased sleep quality, bloating, mood swings, irritability, dry skin and vaginal dryness. The compositions and methods may reduce frequency and/or severity of these symptoms.

In embodiments, provided herein is a method of treating a symptom of menopause, comprising administering to a female a composition comprising (a) a therapeutically effective amount of a compound selected from the group consisting of nicotinamide riboside (NR), nicotinamide mononucleotide (NMN), nicotinamide (NAM), nicotinic acid (NA), the reduced form of nicotinamide riboside (NRH), and a mixture thereof, and (b) a therapeutically effective amount of pterostilbene (PT), wherein the female has one or more symptoms of menopause selected from the group consisting of hot flashes, decreased sleep quality, bloating, mood swings, irritability, dry skin, and vaginal dryness.

In additional embodiments of the method, the composition is administered to the female once per day for at least 7 days.

In other embodiments of the method, the female's estradiol level is higher than baseline for at least 2 consecutive days during the treatment period.

In yet other embodiments of the method, the female's estradiol level is at least 10% higher than baseline for at least 2 consecutive days during the treatment period, and in some embodiments, an increase in the female's estradiol level from baseline occurs on or by day 7 of the administration of the composition.

### BRIEF DESCRIPTION OF THE DRAWINGS

FIGS. 1A-1C depict bar graphs representing pre-, peri-, and post-menopausal subjects (combined) regarding the frequency and unpleasantness of bloating (a), hot flashes (b) and poor sleep (c) compared with baseline (pre-BASIS; prior to treatment).

FIGS. 2A-2C depict bar graphs comparing the effect of NRPT by symptomatic subjects and asymptomatic subjects regarding the frequency and unpleasantness of bloating (a), hot flashes (b) and poor sleep (c) compared with baseline (pre-BASIS; prior to treatment).

FIGS. 3A-3B depict: A-Estradiol levels in urine measured before and after 7 day supplementation with BASIS. B-Estradiol/Estrone (E2/E1) ratios calculated from concentrations of estradiol and estrone measured in urine samples collected before and after 7 day BASIS supplementation.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. Thus, “a”, “an” or “the”, means one or more, unless specified otherwise. For example, treating “a symptom” means treating one or more symptoms.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, “and/or” refers to and encompasses each of the listed items individually, as well as any and all possible combinations of one or more of the listed items.

In addition, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art, all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 members refers to groups having 1, 2, or 3 members. Similarly, a group having 1-5 members refers to groups having 1, 2, 3, 4, or 5 members, and so forth.

Technical and scientific terms used herein have the meaning commonly understood by one of skill in the art to which the present application pertains, unless otherwise defined. Reference is made herein to various methodologies and materials known to those of skill in the art.

An embodiment of the invention provided herein is a method of treating a symptom of menopause, comprising administering to a female a composition comprising (a) a therapeutically effective amount of a compound selected from the group consisting of nicotinamide riboside (NR), nicotinamide mononucleotide (NMN), nicotinamide (NAM), nicotinic acid (NA), the reduced form of nicotinamide riboside (NRH), and a mixture thereof, and (b) a therapeutically effective amount of pterostilbene, wherein the female has one or more symptoms of menopause selected from the group consisting of hot flashes, decreased sleep quality (poor sleep), bloating, mood swings, irritability, dry skin, and vaginal dryness. The methods described herein suitably “treat” a symptom of menopause, which means that the methods and compositions result in the reduction of the severity and/or frequency of a symptom of menopause in the female and can include the complete elimination of the symptom of menopause. Thus, a decrease in severity or decrease in frequency of a symptom means that the symptom may decrease in either severity or frequency, or both in severity and frequency.

The compositions of the invention may be administered to the female periodically, for example, once per day, twice per day, three times per day, every other day, or once per week. The compositions may be administered for a predetermined treatment period or indefinitely. For example, the composition may be administered for 5, 6, 7, 10, 14, 21, 28, 30, 60, 90, 180 or 365 days, or indefinitely. Day 1 is the first day on which the composition is administered to the female.

The female to whom the composition is administered may suffer from vasomotor symptoms of menopause, which may include hot flashes, decreased sleep quality, bloating, mood swings, irritability, dry skin, and/or vaginal dryness, and administration of the composition may result in a decrease in severity or a decrease in frequency of at least one menopausal symptom in the female on or by day 7, 10, 14, 21, or 30 of the treatment period. The treatment period is the period of time during which the female is administered the compositions of the method on a regular basis, such as daily. It is anticipated that on occasion a dose may be missed.

In some embodiments, the decrease in severity or decrease in frequency of at least one menopausal symptom in the female occurs after once daily administration of the composition during the treatment period. In other embodiments, the decrease occurs after twice daily administration.

Suitably, the female's estradiol level is higher than baseline following treatment with BASIS (125 mg nicotinamide riboside and 25 mg pterostilbene) for 7 days, suitably for at least 2 consecutive days during the treatment period. More suitably, the female's estradiol level is higher than baseline following treatment with two doses of BASIS (250 mg nicotinamide riboside and 50 mg pterostilbene) for 7 days, suitably for at least 2 consecutive days during the treatment period. Furthermore, in some embodiments of the methods of the invention the female's estradiol level is at least 10% higher than baseline following treatment with BASIS (one or two doses) for 7 days, suitably for at least 2 consecutive days during the treatment period. Suitably, the increase in the female's estradiol level from baseline occurs on or by day 7 of the treatment period (the period over which the patient is receiving the therapeutically effective amount of the compounds described herein). In other embodiments of the methods of the invention, the female's estradiol level is at least 15% higher than baseline for at least 2 consecutive days during the treatment period, suitably at least 20%, at least 25%, at least 30%, at least 35%, at least 40%, at least 45% or at least 50% higher, including at least 60% higher than baseline following treatment with BASIS (one or two doses), suitably for at least 2 consecutive days during the treatment period.

The female's baseline estradiol level is determined by evaluating the amount of estradiol in a urine sample from the female before day 1 of the treatment period, and in embodiments can be taken as an average estradiol level over a period of 2 or more days, measured before day 1 of the treatment period. In some embodiments, baseline is determined within 7 days before commencement of treatment, or can be determined after the 7 day treatment.

In some embodiments, the female's estradiol level may be evaluated by determining the level of estradiol in the female's urine. For example, a 10 mL urine sample may be analyzed for total estradiol content (estrone, estradiol, estriol, and their sulfate and glucoronate metabolites).

Currently no standardized, knowledge-based, metabolic (i.e., endogenous production) treatment is available for the menopausal symptoms afflicting women over decades of their lives. The invention provides a treatment protocol for maintaining estradiol hormone levels for longer naturally.

Hormone replacement therapy has the potential to promote cancer if dosages are not carefully monitored. Natural means (vitamin B3 enabled NAD levels' boosting) to retain endogenous production of hormones like estradiol is more likely to be effective in addressing some menopause discomfort while less likely to result in excess circulating hormones.

Some embodiments of the invention may result in an increase in endogenous estradiol levels in women.

In other embodiments, the Estradiol/Estrone (E2/E1) ratio calculated from concentrations of estradiol and estrone measured in a female's urine is also increased as a result of the treatment methods described herein. It is has been found, utilizing the methods provided, that a higher E2/E1 ratio favors a reduction in the symptoms of menopause. In exemplary embodiments, the E2/E1 ratios measured following the treatment regimens and periods described herein (suitably, administration with BASIS (one dose or two doses) for at least 7 days) increases the E2/E1 ratio by about 2-fold or about 3-fold, or about 4-fold or more, over baseline. For example, the E2/E1 ratio is suitably increased from about 0.2 to about 0.5 or more, about 0.6 or more, about 0.7 or more, including about 0.8 or more, about 0.9 or more or about 1.0 or more.

Various routes of administration of the compositions described herein may be used to deliver the compositions described herein to treat of a symptom of menopause according to the method of the invention. For example, the compositions can be administered orally to the female, such as in the form of a liquid, drops, a spray, a solution, a gel, a powder, a suspension, or in a solid dosage form. Solid dosage forms include tablets, capsules, pills, troches, gel-caps, lozenges, buccal or sub-lingual strips, cachets, pellets, powders, or granules. Suitably the compositions are delivered orally in the form of gel-caps comprising the compounds described herein.

Additional methods of administration include injection (e.g., via syringe and needle) or infusion (e.g., via venous infusion, including the use of an intravenous infusion bag, needle and required tubing, etc.). Further routes of administration include parenteral, enteral, intranasal, topical, transdermal, intraepidermal, intradermal, subdermal, etc.

Administration of the compositions topically to or into the skin can be accomplished by any well-known means, including in the form of a solution, an ointment, a salve, a patch, a cream, a topical solution, or a depot. Parenteral administration of the compositions described herein can be accomplished via a route such as subcutaneous injection, intravenous infusion, intraarterial infusion, transdermal diffusion, implantation of a drug eluting wafer or film, sublingually, buccally, orally, via aerosol inhalation, intravaginally, rectally, or intracranially.

### Compositions and Formulations for Use in the Methods

In exemplary embodiments, the compositions for use in the methods of the invention comprise nicotinamide riboside (NR), nicotinamide mononucleotide (NMN), nicotinamide (NAM), nicotinic acid (NA), the reduced form of nicotinamide riboside (NRH), or a mixture thereof. A mixture thereof means a mixture of any two or more of NR, NMN, NAM, NA and NRH.

In some embodiments the compositions may contain nicotinamide riboside (NR), a precursor of coenzyme NAD⁺, which is involved in metabolic processes such as energy production, DNA repair, cellular detoxification, the inflammatory response, and protein folding. The chemical structure of NR is provided below.

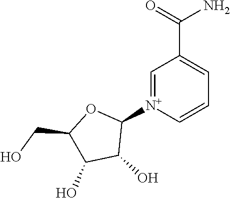 [SMILES: NC(=O)c1ccc[n+]([C@@H]2O[C@H](CO)[C@@H](O)[C@H]2O)c1]

NR has four asymmetric centers and that any optical isomer, as separated, pure or partially purified optical isomers and any mixtures thereof including racemic mixtures can be used. The enantiomeric form can be in enantiomeric excess, e.g., essentially in a pure form. Accordingly, some embodiments relate to NR having an enantiomeric excess of at least 60%, at least 70%, at least 80%, at least 85%, at least 90%, at least 96%, at least 98%, and ranges therebetween.

NR is a quaternary salt and forms an ionic bond with a counter anion. Examples of counter anions include the anions of suitable organic acid such as formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid counter anions include the pharmaceutically acceptable salts listed in J. Pharm. Sci.66, 2 (1977)).

In certain other embodiments, the active agent is a derivative, salt, solvate, or prodrug of NR. In some embodiments, the ribose in NR is β-D-ribose.

As discussed herein, in embodiments, the compositions may contain nicotinamide mononucleotide (NMN), an intermediate in NAD+ biosynthesis. The chemical structure of NMN is provided below.

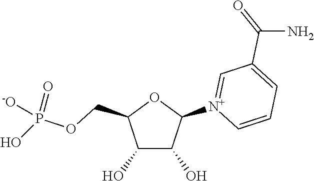

In certain embodiments, the active agent is a derivative, salt, solvate, or prodrug of NMN.

As discussed herein, in embodiments, the compositions may contain nicotinamide (NAM). NAM is also known as niacinamide or nicotinic acid amide, and is the water-soluble, active form of vitamin B3. NAM is incorporated into NAD+ and nicotinamide adenine dinucleotide phosphate (NADP+), which are cofactors in some enzymatic oxidation-reduction reactions. The chemical structure of NAM is provided below.

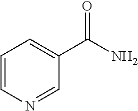

In certain embodiments, the active agent is a derivative, salt, solvate, or prodrug of NAM.

As discussed herein, in embodiments, the compositions may contain nicotinic acid (NA), which is also known as niacin or vitamin B3. The amide derivative of NA, NAM, is a component of NAD and NADP+. The chemical structure of NA is provided below.

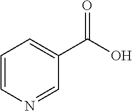

In certain embodiments, the active agent is a derivative, salt, solvate, or prodrug of NA.

As discussed herein, in embodiments, the compositions may contain the reduced form of nicotinamide riboside (NRH). NRH is a potent NAD+ precursor in cells. NRH leads to NAD+ synthesis through a path independent of that of NR. The chemical structure of NRH is provided below.

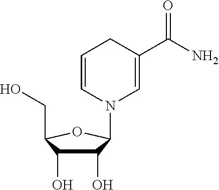

In certain embodiments, the active agent is a derivative, salt, solvate, or prodrug of NRH.

Suitably, the compositions also include pterostilbene. Pterostilbene is a polyphenol based derivative of resveratrol and promotes metabolic health. The chemical structure of pterostilbene is provided below:

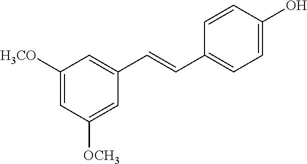 [SMILES: COc1cc(/C=C/c2ccc(O)cc2)cc(OC)c1]

In some embodiments, a derivative, salt, solvate, or prodrug of pterostilbene can be used in the compositions described herein. In certain embodiments, pterostilbene may be substituted and/or combined with epsilon-viniferin and/or resveratrol.

NR, NMN, NAM, NA, NRH, and pterostilbene, or derivatives thereof, may be chemically modified so that oral delivery of the compound is efficacious. Contemplated chemical modification is the attachment of at least one moiety to the component molecule itself, where the moiety permits uptake into the blood stream from the stomach or intestine, or uptake directly into the intestinal mucosa. Also contemplated is the increase in overall stability of the component or components and increase in circulation time in the body.

In suitable embodiments, the NR, NMN, NAM, NA, NRH, or mixture thereof, is contained within the same composition as the pterostilbene. In other embodiments, the pterostilbene is in a different composition than the NR, NMN, NAM, NA, NRH, or mixture thereof.

As described herein, the compositions for use in the treatment methods are suitably formulated for oral delivery, i.e., in an oral formulation. The formulation is suitably an oral formulation, including a liquid, drops, a spray, a solution, a gel, a powder, a suspension, or in a solid dosage form.

Oral solid dosage forms are described generally in Remington's Pharmaceutical Sciences, 18th Ed. 1990 (Mack Publishing Co. Easton Pa. 18042), Chapter 89. Solid dosage forms include tablets, capsules, pills, troches, gel-caps, lozenges, buccal or sub-lingual strips, cachets, pellets, powders, or granules or incorporation of the material into particulate preparations of polymeric compounds such as polylactic acid, polyglycolic acid, etc., or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also, Marshall, K. In: Modern Pharmaceutics Edited by G. S. Banker and C. T. Rhodes Chapter 10, 1979. The formulation may include a peptide (or chemically modified forms thereof) and inert ingredients which protect compounds in the stomach environment, and release of the biologically active material in the intestine.

Certain embodiments provide liquid dosage forms for oral administration, including pharmaceutically acceptable emulsions, solutions, suspensions, and syrups, which may contain other components including inert diluents, adjuvants such as wetting agents, emulsifying and suspending agents, and sweetening, and flavoring agents. The methods can involve the use of a composition which is administered as a liquid with an active agent (e.g., NR, NMN, NAM, NA, NRH, pterostilbene, derivatives of these compounds, and/or mixtures of any of these compounds or derivatives) dissolved (e.g., solution) or dispersed (e.g., suspension) in the composition. The solution or suspension may be prepared using one or more pharmaceutically acceptable excipients. Suitable excipients include, but are not limited to, surfactants, humectants, plasticizers, crystallization inhibitors, wetting agents, bulk filling agents, solubilizers, bioavailability enhancers, pH adjusting agents, flavorants and combinations

Controlled release oral formulations may be provided. Controlled release may include, but is not limited to, delayed release and pH-dependent release. In certain embodiments, the active agent can be incorporated into microcapsules, microparticulates, nanoparticulates, etc. through use of coatings to affect release of the active agent/s. In certain embodiments, the active agent/s can be incorporated into an inert matrix which permits release by either diffusion or leaching mechanisms, e.g., gums. Slowly degenerating matrices may also be incorporated into the formulation.

Modified release oral formulations may be provided. Modified release may allow for specific release profiles.

Extended release oral formulations may be provided. Extended release may allow for release of active agent over a desired time period.

Additional discussion of varying release formulations and related terms may be found in Lesczek Krowczynski, Extended-Release Dosage Forms, 1987 (CRC Press, Inc.).

In certain aspects, the form of a controlled, modified or extended release oral formulation is a tablet, capsule, or microbeads for oral administration. In other aspects, controlled, modified or extended release formulations comprising suitable and effective treatment amounts of the desired components may be pills, powders, granules, sterile parenteral solutions or suspensions, oral solutions or suspensions, oil-in-water emulsions as well as implants and microencapsulated delivery systems.

Other formulations may provide controlled, modified or extended release profiles. Compositions of the present invention may comprise conventional pharmaceutical binders, excipients and additives, which may act to control, modify or extend release when used in sufficient quantities. Coating agents, e.g., plasticizers, may be used to enhance the controlled, modified or extended release features of the compositions of the invention.

For oral formulations, the location of release may be the stomach, the small intestine (the duodenum, the jejunum, or the ileum), or the large intestine. The release can avoid the deleterious effects of the stomach environment, either by protection of the agent or by release of the agent beyond the stomach environment, such as in the intestine. To ensure full gastric resistance a coating temporally impermeable to at least pH 5.0 is useful. Examples of the more common inert ingredients that are used as enteric coatings are cellulose acetate trimellitate (CAT), hydroxypropylmethylcellulose phthalate (HPMCP), polyvinyl acetate phthalate (PVAP), poly(methacrylic acid-co-ethyl acrylate) 1:1, cellulose acetate phthalate (CAP), poly(methacylic acid-co-methyl methacrylate) 1:1, poly(methacylic acid-co-methyl methacrylate) 1:2, and natural shellac resin. These coatings may be used as mixed films.

In exemplary embodiments, the methods described herein utilize the oral administration of soft capsules containing NR, NMN, NAM, NA, NRH, mixtures thereof, and pterostilbene, or their equivalents. The soft capsule can be prepared using techniques well known in the art. For example, soft capsules are typically produced using a rotary die encapsulation process. Active agent formulations are fed into the encapsulation machine by gravity. In an embodiment, the formulation comprises pharmaceutical excipients such as olive oil, gelatin, glycerin, purified water, beeswax yellow, sunflower lecithin, silicon dioxide, titanium dioxide, F. D. & C Blue 1 and F. D. & C Red 4, microcrystalline cellulose, hypromellose, vegetable magnesium stearate, and/or silica.

A capsule shell can comprise one or more plasticizers such as glycerin, sorbitol, sorbitans, maltitol, glycerol, polyethylene glycol, polyalcohols with 3 to 6 carbon atoms, citric acid, citric acid esters, triethyl citrate and combinations thereof. In an embodiment, the plasticizer is glycerin.

In addition to the plasticizer(s), the capsule shell can include other suitable shell additives such as opacifiers, colorants, humectants, preservatives, flavorings, and buffering salts and acids.

Opacifiers are used to opacify the capsule shell when the encapsulated active agents are light sensitive. Suitable opacifiers include, but are not limited to, titanium dioxide, zinc oxide, calcium carbonate and combinations thereof. In an embodiment, the opacifier is titanium dioxide.

Colorants can be used to for marketing and product identification and/or differentiation purposes. Suitable colorants include synthetic and natural dyes and combinations thereof.

Humectants can be used to suppress the water activity of the softgel. Suitable humectants include glycerin and sorbitol, which are often components of the plasticizer composition. Due to the low water activity of dried, properly stored softgels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

One composition for use in the methods of treatment described herein is referred to as “BASISR” and includes nicotinamide riboside (125 mg/capsule) and pterostilbene (25 mg/capsule) as the active agents. This can be in a capsule formed of microcrystalline cellulose, hypromellose, vegetable magnesium stearate, olive oil, gelatin, glycerin, purified water, beeswax yellow, sunflower lecithin, silicon dioxide, titanium dioxide, F. D. & C Blue 1 and F. D. & C Red 4, or vegetarian hard capsules made solely of plant materials. Any embodiment may include microcrystalline cellulose, hypromellose, vegetable magnesium stearate, and/or silica. Other pharmaceutical excipients that can be included in the formulations, include acetyl-L-carnitine, N-acetyl cysteine, α-lipoic acid, biotin, vitamin B6, vitamin B12, folic acid, resveratrol, vinpocetine, chromium picolinate, vitamin D3, naringin, quercetin, and creatine.

Formulations that can be administered as part of a depot, wafer, dissolvable matrix, etc., suitably include pharmaceutically acceptable polymers, solidifying agents, cake forming agents, etc.

Other suitable formulations include injectable formulation or intravenously administered formulations or infusion formulations, and may include various excipients, such as aqueous buffers, salts, pH modifying agents, osmolarity modifying agents, preservatives, antimicrobial agents, etc., and suitably include pharmaceutically acceptable carriers such as saline-based carriers, etc. “Injectable formulations” or “infusion formulations” are liquid formulations that can be prepared and included in a syringe, pump, drip bag, single dose injector, etc., for direct injection into a patient or for infusion intravenously into the patient.

Other suitable formulations include topical formulations. As used herein “topical formulation” refers to any form of a composition or formulation that can be applied to the surface of the skin, including the scalp, face, eyebrows, eyelashes, back, neck, trunk, legs, etc. Topical formulations include liquids, foams, powders, emulsions, sprays, creams, lotions, serums, salves, balms, etc. Examples of inactive ingredients that can be included in topical formulations include moisturizers, humectants, odor modifiers, buffers, pigments, preservatives, Vitamins such as A, C and E, emulsifiers, dispersing agents, wetting agents, odor-modifying agents, gelling agents, stabilizers, propellants, antimicrobial agents, sunscreens, enzymes and the like.

As used herein “therapeutically effective” refers to the amount of NR, NMN, NAM, NA, NRH, and/or pterostilbene needed to treat a symptom of menopause. In certain embodiments, a mixture of NR, NMN, NAM, NA and/or NRH may be used. In certain embodiments epsilon-viniferin and/or resveratrol may be substituted for pterostilbene. In certain embodiments, a combination of pterostilbene, epsilon-viniferin, and/or resveratrol maybe used.

In exemplary embodiments the total dose administered per day of the compound NR, NMN, NAM, NA, NRH, or mixture thereof, is an amount between about 1 mg and about 1500 mg, between about 5 mg and about 1500 mg, between about 10 mg and about 1000 mg, between about 10 mg and about 900 mg, between about 10 mg and about 850 mg, between about 50 mg and about 700 mg, between about 50 mg and about 500 mg, between about 50 mg and about 300 mg, between about 50 mg and about 250 mg, between about 100 mg and about 700 mg, between about 100 mg and about 500 mg, between about 150 mg and about 500 mg, between about 200 mg and about 500 mg, between about 1000 mg and about 1500 mg, or is about 5 mg, about 10 mg, about 20 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg or about 700 mg. Where the compound is a mixture of one or more of NR, NMN, NAM, NA, or NRH, the weight of the components of the mixture are added together to achieve the total daily dose.

Suitably, these amounts are administered on a daily basis in the form of a single composition or the total daily dose can be divided into two or more separate compositions (e.g., a daily dose of 250 mg can be given in two separate doses of 125 mg, e.g., two capsules of BASIS each containing 125 mg). These separate compositions can be administered at the same time, or at different times during the day, e.g., morning and evening. The administration of the compound can be via any administration route described herein. As noted above, the NR, NMN, NAM, NA, NRH, or mixture thereof, can be administered in the same composition with pterostilbene or its equivalents, or can be in a composition separate from the pterostilbene or its equivalents.

In exemplary embodiments, the total dose administered per day of pterostilbene or its equivalents, is an amount between about 25 mg and about 1000 mg, between about 100 mg and about 1000 mg, between about 25 mg and about 500 mg, between about 25 mg and about 200 mg, between about 25 mg and about 250 mg, between about 30 mg and about 225 mg, between about 40 mg and about 200 mg, between about 45 mg and about 250 mg, or is about 25 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg pterostilbene.

Suitably, these amounts are administered on a daily basis in the form of a single composition or the total daily dose can be divided into two or more separate compositions (e.g., a daily dose of 50 mg can be given in two separate doses of 25 mg, e.g., two capsules of BASIS each containing 25 mg). These separate compositions can be administered at the same time, or at different times during the day, e.g., morning and evening. The administration of pterostilbene can be via any administration route described herein. As noted above, the pterostilbene or its equivalents can be administered in the same composition with the NR, NMN, NAM, NA, NRH, or mixture thereof, or can be administered in a separate composition from the NR, NMN, NAM, NA, NRH, or mixture thereof.

Suitably, the methods of treatment described herein include administering a composition comprising NR at about 100 mg to about 350 mg and pterostilbene at about 10 mg to about 100 mg. In some embodiments, the methods of treatment include administering two or more doses of such a composition, such that a total of about 200 mg to about 700 mg NR and about 20 mg to about 200 mg pterostilbene is administered on a daily basis. In other embodiments, the methods of treatment include administering two or more doses of the composition, such that a total of about 250 mg NR and about 50 mg pterostilbene is administered on a daily basis. The administration of the composition can be via any administration route described herein.

Suitably, the methods of treatment described herein include administering a composition comprising NMN at about 100 mg to about 350 mg and pterostilbene at about 10 mg to about 100 mg. In some embodiments, the methods of treatment include administering two or more doses of such a composition, such that a total of about 200 mg to about 700 mg NMN and about 20 mg to about 200 mg pterostilbene is administered on a daily basis. In other embodiments, the methods of treatment include administering two or more doses of the composition, such that a total of about 200 to about 250 mg NMN and about 50 mg pterostilbene is administered on a daily basis. The administration of the composition can be via any administration route described herein.

Suitably, the methods of treatment described herein include administering a composition comprising NAM at about 5 mg to about 350 mg and pterostilbene at about 10 mg to about 100 mg. In some embodiments, the methods of treatment include administering two or more doses of such a composition, such that a total of about 10 mg to about 700 mg NAM and about 20 mg to about 200 mg pterostilbene is administered on a daily basis. In other embodiments, the methods of treatment include administering two or more doses of the composition, such that a total of about 20 to about 200 mg NAM and about 50 mg pterostilbene is administered on a daily basis. The administration of the composition can be via any administration route described herein.

Suitably, the methods of treatment described herein include administering a composition comprising NA at about 5 mg to about 350 mg and pterostilbene at about 10 mg to about 100 mg. In some embodiments, the methods of treatment include administering two or more doses of such a composition, such that a total of about 10 mg to about 700 mg NA and about 20 mg to about 200 mg pterostilbene is administered on a daily basis. In other embodiments, the methods of treatment include administering two or more doses of the composition, such that a total of about 20 to about 200 mg NA and about 50 mg pterostilbene is administered on a daily basis. The administration of the composition can be via any administration route described herein.

Suitably, the methods of treatment described herein include administering a composition comprising NRH at about 100 mg to about 350 mg and pterostilbene at about 10 mg to about 100 mg. In some embodiments, the methods of treatment include administering two or more doses of such a composition, such that a total of about 200 mg to about 700 mg NRH and about 20 mg to about 200 mg pterostilbene is administered on a daily basis. In other embodiments, the methods of treatment include administering two or more doses of the composition, such that a total of about 200 to about 250 mg NRH and about 50 mg pterostilbene is administered on a daily basis. The administration of the composition can be via any administration route described herein.

In certain embodiments, a composition may be administered for a period of days, weeks, or months. The composition may be administered once per day, twice per day, or multiple times per day. When the compositions are administered for multiple days, weeks, or months, the dose per day may not be exactly the same each day of the treatment period. For example, one dose may be given for an initial period of several days, and then the dose may be increased after this initial period. Alternatively, if a composition is administered more than once per day, the dose may not be the same each time the composition is administered in a given day. Dosage amounts during a dosage regimen may vary according to the amounts and ranges disclosed herein.

Suitably, the compositions described herein are administered on a once daily basis for a period of at least 1 week, at least 4 weeks, at least 1 month, or for a period of at least 2 months, at least 3 months, at least 4 months, at least 5 months, at least 6 months, at least 30 days, at least 60 days, at least 90 days, at least 120 days, at least 150 days, at least 180 days, at least 210 days, at least 240 days, at least 270 days, at least 300 days, at least 330 days, at least 360 days, etc. The compositions described herein can also be administered for 1 or more years, including for the lifetime of the female. The dosage can be provided via any administration route, as described herein.

In compositions and formulations that “consist essentially” of the recited ingredients, such compositions and formulations contain the recited components and those that do not materially affect the basic and novel characteristics of the claimed formulations. Components that do not materially affect the basic and novel characteristics of the claimed formulations are those that do not limit the ability of the NR, NMN, NAM, NA, NRH, or mixture thereof, or of the pterostilbene, to treat the symptom of menopause. Suitably, compositions and formulations that consist essentially of the recited ingredients specifically exclude other active agents for treatment of symptoms of menopause.

As described herein, suitably the compositions comprise a combination of NR at about 100 mg to about 350 mg and pterostilbene at about 10 mg to about 100 mg, and more suitably the compositions comprise a combination of NR at about 125 mg and pterostilbene at about 25 mg (i.e., BASIS®). In embodiments, two doses of BASIS are utilized, providing a combination of NR at about 250 mg and pterostilbene at about 50 mg.

In other embodiments the compositions suitably comprise a combination of NMN at about 100 mg to about 350 mg and pterostilbene at about 10 mg to about 100 mg, and more suitably comprise a combination of NMN at about 100 to about 125 mg and pterostilbene at about 25 mg.

In other embodiments the compositions suitably comprise NAM at about 5 mg to about 350 mg and pterostilbene at about 10 mg to about 100 mg, and more suitably comprise a combination of NAM at about 10 mg to about 100 mg and pterostilbene at about 25 mg.

In yet other embodiments the compositions suitably comprise NA at about 5 mg to about 350 mg and pterostilbene at about 10 mg to about 100 mg, and more suitably comprise a combination of NA at about 10 mg to about 100 mg and pterostilbene at about 25 mg.

In still other embodiments the compositions suitably comprise NRH at about 100 mg to about 50 mg and pterostilbene at about 10 mg to about 100 mg, and more suitably comprise a combination of NRH at about 100 mg to about 125 mg and pterostilbene at about 25 mg.

In exemplary embodiments, the compositions are administered orally to the female. In other embodiments, administration can be via injection or infusion. Additional routes of administration include administration parenterally, enterally, or transdermally, to the patient.

### Exemplary Embodiments

Embodiment 1 is a method of treating a symptom of menopause, comprising administering to a female a composition comprising (a) a therapeutically effective amount of a compound selected from the group consisting of NR, NMN, NAM, NA, NRH, and a mixture thereof, and (b) a therapeutically effective amount of pterostilbene, wherein the female has one or more symptoms of menopause selected from the group consisting of hot flashes, decreased sleep quality, bloating, mood swings, irritability, dry skin, and vaginal dryness.

Embodiment 2 includes the method of embodiment 1, wherein the female suffers from vasomotor symptoms of menopause.

Embodiment 3 includes the methods of embodiments 1 or 2, wherein the female suffers from decreased sleep quality.

Embodiment 4 includes the methods of any of embodiments 1-3, wherein the composition is administered to the female once per day for at least 7 days.

Embodiment 5 includes the methods of any of embodiments 1-4, wherein the female's estradiol level is higher than baseline for at least 2 consecutive days during the treatment period.

Embodiment 6 includes the method of embodiment 5, wherein the female's estradiol level is at least 30% higher than baseline following the treatment period.

Embodiment 7 includes the method of embodiment 4, wherein an increase in the female's estradiol level from baseline occurs on or by day 7 of the administration of the composition.

Embodiment 8 includes the methods of any of embodiments 4-7, wherein there is a decrease in severity of at least one menopausal symptom in the female on or by day 7 of the administration of the composition.

Embodiment 9 includes the methods of any of embodiments 4-7, wherein there is a decrease in frequency of at least one menopausal symptom in the female on or by day 7 of the administration of the composition.

Embodiment 10 includes the methods of any of embodiments 1-9, wherein the composition is administered parenterally, enterally, intranasally, or transdermally.

Embodiment 11 includes the methods of any of embodiments 1-9, wherein the composition is administered orally.

Embodiment 12 includes the methods of any of embodiments 1-11, wherein the amount of the compound administered per day is about 10 mg to about 700 mg and the amount of pterostilbene administered per day is about 25 mg to about 200 mg.

Embodiment 13 includes the methods of any of embodiments 1-11, wherein the amount of the compound administered per day is about 50 mg to about 250 mg and the amount of pterostilbene administered per day is about 50 mg.

Embodiment 14 is a method of treating a symptom of menopause, comprising orally administering to a female for at least 7 days a composition comprising NR and pterostilbene, wherein the amount of NR administered per day is about 200 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

Embodiment 15 includes the method of embodiment 14, wherein the amount of NR administered per day is about 250 mg and the amount of pterostilbene administered per day is about 50 mg.

Embodiment 16 includes the methods of embodiments 14 or 15, wherein the female's estradiol level is higher than baseline for at least 2 consecutive days during the treatment period.

Embodiment 17 includes the method of embodiment 16, wherein the female's estradiol level is at least 30% higher than baseline following the treatment period.

Embodiment 18 is a method of treating a symptom of menopause, comprising orally administering to a female for at least 7 days a composition comprising NMN and pterostilbene, wherein the amount of NMN administered per day is about 200 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

Embodiment 19 includes the method of embodiment 18, wherein the amount of NMN administered per day is about 200 to about 250 mg and the amount of pterostilbene administered per day is about 50 mg.

Embodiment 20 includes the methods of embodiments 18 or 19, wherein the female's estradiol level is higher than baseline for at least 2 consecutive days during the treatment period.

Embodiment 21 includes the methods of embodiment 20, wherein the female's estradiol level is at least 30% higher than baseline following the treatment period.

Embodiment 22 is a method of treating a symptom of menopause, comprising orally administering to a female for at least 7 days a composition comprising NAM and pterostilbene, wherein the amount of NAM administered per day is about 10 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

Embodiment 23 includes the method of embodiment 22, wherein the amount of NAM administered per day is about 20 mg to about 200 mg and the amount of pterostilbene administered per day is about 50 mg.

Embodiment 24 includes the methods of embodiments 22 or 23, wherein the female's estradiol level is at least 30% higher than baseline following the treatment period.

Embodiment 25 includes the method of embodiment 24, wherein the female's estradiol level is at least 50% higher than baseline following the treatment period.

Embodiment 26 is a method of treating a symptom of menopause, comprising orally administering to a female for at least 7 days a composition comprising NA and pterostilbene, wherein the amount of NA administered per day is about 10 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

Embodiment 27 includes the method of embodiment 26, wherein the amount of NA administered per day is about 20 mg to about 200 mg and the amount of pterostilbene administered per day is about 50 mg.

Embodiment 28 includes the methods of embodiments 26 or 27, wherein the female's estradiol level is at least 30% higher than baseline following the treatment period.

Embodiment 29 includes the method of embodiment 28, wherein the female's estradiol level is at least 50% higher than baseline following the treatment period.

Embodiment 30 is a method of treating a symptom of menopause, comprising orally administering to a female for at least 7 days a composition comprising NRH and pterostilbene, wherein the amount of NRH administered per day is about 200 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

Embodiment 31 includes the method of embodiment 30, wherein the amount of NRH administered per day is about 200 mg to about 250 mg and the amount of pterostilbene is about 50 mg.

Embodiment 32 includes the methods of embodiments 30 or 31, wherein the female's estradiol level is at least 30% higher than baseline following the treatment period.

Embodiment 33 includes the method of embodiment 32, wherein the female's estradiol level is at least 50% higher than baseline following the treatment period.

Embodiment 34 is a composition comprising a combination of (a) a compound selected from the group consisting of: NR, NMN, NAM, NA, NRH, and a mixture thereof, at about 5 mg to about 350 mg and (b) pterostilbene at about 10 mg to about 100 mg, for use in treating a symptom of menopause in a female, wherein the symptom treated is hot flashes, decreased sleep quality, bloating, mood swings, irritability, dry skin or vaginal dryness.

### EXAMPLES

### Example 1: Reducing the Severity and/or Frequency of Menopausal Symptoms Using Nicotinamide Riboside and Pterostilbene

A single arm study was performed to determine whether supplementation with a composition containing nicotinamide riboside (NR) and pterostilbene (PT) would increase the natural production of estradiol, measured in urinary waste. The secondary objective was to determine whether undesirable menopausal effects, assessed via questionnaires, would be mitigated by a short-term supplementation with the NRPT composition. The composition was an encapsulated mixture of synthetic nicotinamide riboside and pterostilbene (NRPT), commercially known as BASIS.

Forty (40) healthy women over 35 years of age were enrolled in this study. Eight (n=8) subjects were pre-menopausal and asymptomatic, so served as a control. Thirty-two (n=32) subjects were symptomatic, and peri-menopausal or post-menopausal. In a study questionnaire given on day 0 and day 8, subjects were asked to report discomfort, such as hot flashes, poor sleep (decreased sleep quality), and bloating. Potential subjects were excluded if they were under 35 year of age, pregnant or lactating, actively participating in another clinical trial, a member of a prison system, currently receiving hormone replacement therapy, currently undergoing active cancer treatment, or currently using an immunosuppressant.

Following completion of the consent form, the subjects were asked to fill a pre-supplementation questionnaire. They then were asked to provide a urine sample after being given a urine sampling kit. The questionnaire sought information on their current state in view of symptoms associated with menopausal discomfort in addition to demographics (age, sex, race, ethnicity). The subject was also supplied with their first supply of the NRPT composition (day 0) (small tub containing 7 days of BASIS capsules). The subjects took 2 capsules once a day (total daily dose contains 250 mg of nicotinamide riboside and 50 mg of pterostilbene). At day 8 (7 days of treatment with BASIS), the subjects were asked to provide a second urine in the urine sampling kit provided to them and to complete the same questionnaire administered at enrollment. The questionnaire aimed to evaluate the severity and frequency of the most common menopause associated undesirable symptoms.

Once collected and de-identified, urine samples were stored in a dedicated −80° C. freezer until all samples were ready to be processed and analyzed. The urine samples (10 mL) were analyzed for total estradiol content (estrone, estradiol, estriol, and their sulfate and glucoronate metabolites) and for total vitamin B3 metabolites content.

### Data Analysis

Urine samples underwent a targeted analysis of the NAD and estradiol metabolomic profile, using ultra high-performance liquid chromatography mass spectrometry.

Survey responses were analyzed to determine demographics, frequency and severity of menopausal symptoms.

### Statistical Analysis

This is a short-term metabolic study for which pharmacokinetics and pharmacodynamics outcomes are anticipated. There is no intention-to-treat methodology in the analysis. There is no sample stratification. The urine content of the NAD and estradiol metabolomes' measurements are presented as mean of biological replicates and 95% confidence interval (CI) from all analyzed LC-MS measurements pooled from 2 technical replicates. For multiple comparisons, ANOVA follows by post hoc test, i.e., Bonferroni correction for LC-MS and Dunnett's or Tukey's, are used for combination with questionnaire outcomes. Dependent t-tests and repeated measures ANOVA are used to evaluate within sub-groups variability. Statistical analysis is performed using GraphPad PRISM 7.

### Outcomes

The primary outcome for this study was the analysis of the frequency and severity of symptoms experienced by subjects over the 7 days of taking the NRPT composition. Components of symptom severity include development of symptoms and severity of each symptom, such as hot flashes, hot sweats at night, bloating, and difficulty sleeping.

Secondary outcomes for this study include a more in-depth analysis of primary outcomes, including changes in the levels of estradiol and NAD metabolites measured from urine samples.

It was expected that the combination of NR and PT would result in a reduction of the subject's menopause symptoms, including reducing the severity or frequency one or more of the following symptoms: hot flashes, poor sleep (decreased sleep quality), bloating, mood swings, irritability, dry skin, and vaginal dryness.

Seven days of treatment with the combination of NR and PT (BASIS) resulted in a statistically significant reduction of both frequency and unpleasantness of bloating, hot flashes and poor sleep in the peri- and post-menopausal subjects, but not in pre-menopausal (control) subjects.

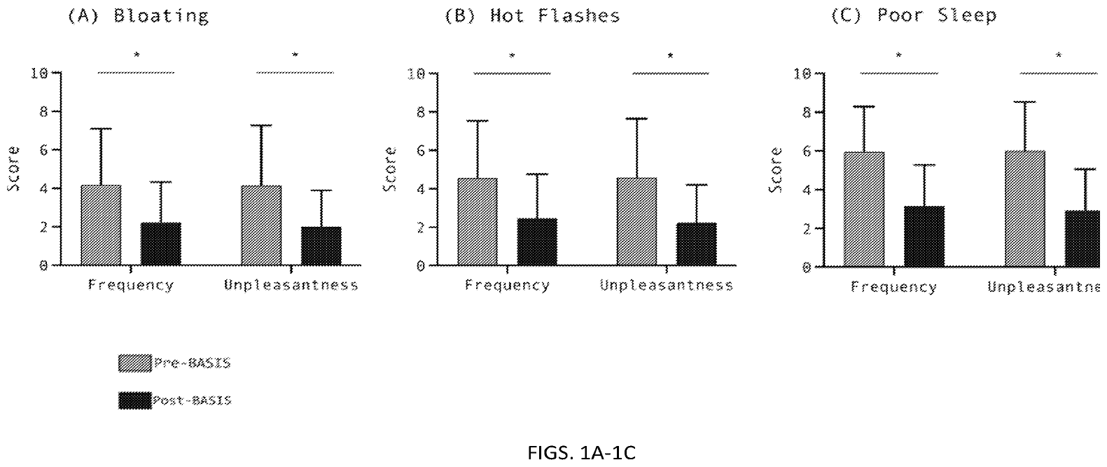

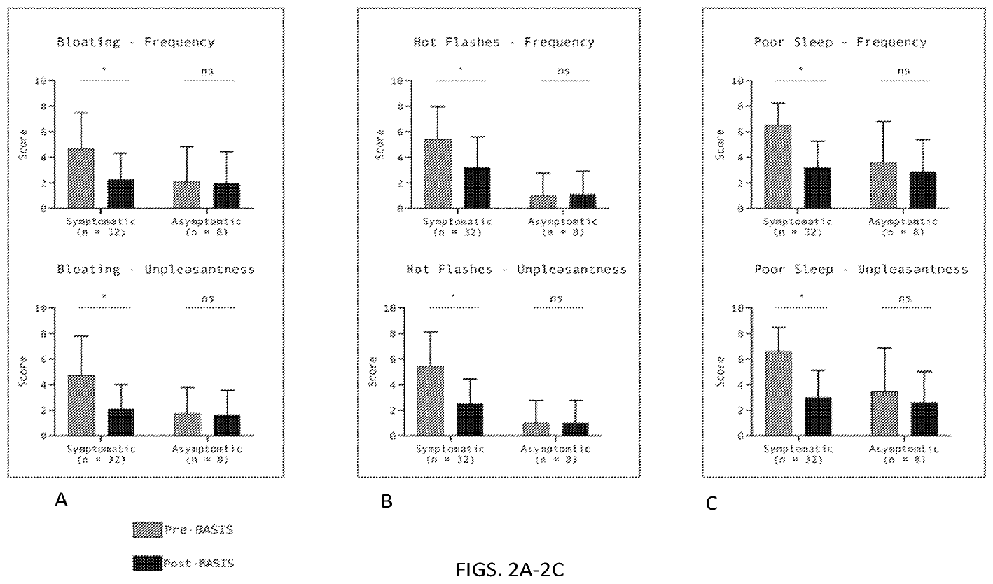

The bar graphs in FIGS. 1A-1C and FIGS. 2A-2C were generated from the subjects' answers to the study questionnaire on days 0 (pre-treatment) and day 8 (post-treatment). The bar graphs in FIGS. 1A-1C compare the pre-treatment and post-treatment reporting by all subjects (peri-, pre- and post-menopausal) in the study regarding the frequency and unpleasantness of bloating (a), hot flashes (b) and poor sleep (c). Treatment resulted in a statistically significant reduction of approximately 50% in both frequency and unpleasantness of all three symptoms. The bar graphs in FIGS. 2A-2C compare these results to the frequency and unpleasantness of pre and post treatment bloating, hot flashes and poor sleep in both the symptomatic subjects (peri- and post-menopausal) and asymptomatic (pre-menopausal/control) subjects. As can be seen from the graphs, treatment had no significant effect on bloating and hot flashes in asymptomatic subjects, and little effect on poor sleep in asymptomatic subjects. However, larger statistically significant reductions are observed in the symptomatic group for all endpoints (compared to FIGS. 1A-1C) now that that the asymptomatic subjects have been separated out.

### Example 2: Reducing the Severity and/or Frequency of Menopausal Symptoms Using Nicotinamide Mononucleotide and Pterostilbene

A single arm study is performed to determine whether supplementation with a composition containing nicotinamide mononucleotide (NMN) and pterostilbene (PT) increases the natural production of estradiol, measured in urinary waste. The secondary objective is to determine whether through increased estradiol levels, the undesirable menopausal effects, assessed via questionnaires, are mitigated by a short-term supplementation with the NMN/PT composition. The composition is an encapsulated mixture of NMN and PT.

Up to 40 healthy women over 35 years of age experiencing undesirable menopause-related discomfort are entered in this study. In a study questionnaire to be given on day 0 and day 8, subjects will be asked to report discomfort including mood-swings, hot flashes, poor sleep, bloating, and dry skin. Potential subjects are excluded if they are under 35 year of age, pregnant or lactating, actively participating in another clinical trial, a member of a prison system, currently receiving hormone replacement therapy, currently undergoing active cancer treatment, or currently using an immunosuppressant.

Following completion of the consent form, the subjects are asked to fill a pre-supplementation questionnaire. They then are asked to provide a urine sample after being given a urine sampling kit. The questionnaire seeks information on their current state in view of symptoms associated with menopausal discomfort in addition to demographics (age, sex, race, ethnicity). The subject is also supplied with their first supply of the NMN/PT composition (day 0) (small tub containing 7 days of capsules). The subject will take 2 capsules once a day (total daily dose contains 250 mg of NMN and 50 mg of PT). At day 8, the subjects are asked to provide a second urine in the urine sampling kit provided to them and fill in the same questionnaire administered at enrollment. The questionnaire aims to evaluate the severity and frequency of the most common menopause associated undesirable symptoms.

Once collected and de-identified, urine samples are stored in a dedicated −80° C. freezer until all samples are ready to be processed and analyzed. The urine samples (10 mL) are analyzed for total estradiol content (estrone, estradiol, estriol, and their sulfate and glucoronate metabolites) and for total vitamin B3 metabolites' content.

### Data Analysis

Urine samples undergo a targeted analysis of the NAD and estradiol metabolomic profile, using ultra high-performance liquid chromatography mass spectrometry.

Survey responses are analyzed to determine demographics, and severity of menopausal symptoms.

### Statistical Analysis

This is a short-term metabolic study for which pharmacokinetics and pharmacodynamics outcomes are anticipated. There will be no intention-to-treat methodology in the analysis. There will be no sample stratification. The urine content of the NAD and estradiol metabolomes' measurements will be presented as mean of biological replicates and 95% confidence interval (CI) from all analyzed LC-MS measurements pooled from 2 technical replicates. For multiple comparisons, ANOVA followed by post hoc test, i.e., Bonferroni correction for LC-MS and Dunnett's or Tukey's for combination with questionnaire outcomes. Dependent t-tests and repeated measures ANOVA will be used to evaluate within sub-groups variability. Statistical analysis is performed using GraphPad PRISM 7.

### Outcomes

The primary outcome for this study is the analysis of severity of symptoms experienced by the individuals over the 7 days of taking the NMN/PT composition. Components of symptom severity include development of symptoms and severity of each symptom, such as hot flashes, hot sweats at night, bloating, and difficulty sleeping.

Secondary outcomes for this study include a more in-depth analysis of primary outcomes, including changes in the levels of estradiol and NAD metabolites measured from urine samples.

It is expected that the combination of NMN and PT results in a reduction of the subject's menopause symptoms, including reducing the severity or frequency one or more of the following symptoms: hot flashes, poor sleep (decreased sleep quality), bloating, mood swings, irritability, dry skin, and vaginal dryness.

### Example 3: Reducing the Severity and/or Frequency of Menopausal Symptoms Using Nicotinamide and Pterostilbene

A single arm study is performed to determine whether supplementation with a composition containing nicotinamide (NAM) and pterostilbene (PT) increases the natural production of estradiol, measured in urinary waste. The secondary objective is to determine whether through increased estradiol levels, the undesirable menopausal effects, assessed via questionnaires, are mitigated by a short-term supplementation with the NAM/PT composition. The composition is an encapsulated mixture of NAM and PT.

Up to 40 healthy women over 35 years of age experiencing undesirable menopause-related discomfort are entered in this study. In a study questionnaire to be given on day 0 and day 8, subjects will be asked to report discomfort includes mood-swings, hot flashes, poor sleep, bloating, and dry skin. Potential subjects are excluded if they are under 35 year of age, pregnant or lactating, actively participating in another clinical trial, a member of a prison system, currently receiving hormone replacement therapy, currently undergoing active cancer treatment, or currently using an immunosuppressant.

Following completion of the consent form, the subjects are asked to fill a pre-supplementation questionnaire. They then are asked to provide a urine sample after being given a urine sampling kit. The questionnaire seeks information on their current state in view of symptoms associated with menopausal discomfort in addition to demographics (age, sex, race, ethnicity). The subject is also supplied with their first supply of the NAM/PT composition (day 0) (small tub containing 7 days of capsules). The subject will take 2 capsules once a day (total daily dose contains 125 mg of NAM and 50 mg of PT). At day 8, the subjects are asked to provide a second urine in the urine sampling kit provided to them and fill in the same questionnaire administered at enrollment. The questionnaire aims to evaluate the severity and frequency of the most common menopause associated undesirable symptoms.

Once collected and de-identified, urine samples are stored in a dedicated −80° C. freezer until all samples are ready to be processed and analyzed. The urine samples (10 mL) are analyzed for total estradiol content (estrone, estradiol, estriol, and their sulfate and glucoronate metabolites) and for total vitamin B3 metabolites' content.

### Data Analysis

Urine samples undergo a targeted analysis of the NAD and estradiol metabolomic profile, using ultra high-performance liquid chromatography mass spectrometry.

Survey responses are analyzed to determine demographics, and severity of menopausal symptoms.

### Statistical Analysis

This is a short-term metabolic study for which pharmacokinetics and pharmacodynamics outcomes are anticipated. There will be no intention-to-treat methodology in the analysis. There will be no sample stratification. The urine content of the NAD and estradiol metabolomes' measurements will be presented as mean of biological replicates and 95% confidence interval (CI) from all analyzed LC-MS measurements pooled from 2 technical replicates. For multiple comparisons, ANOVA followed by post hoc test, i.e., Bonferroni correction for LC-MS and Dunnett's or Tukey's for combination with questionnaire outcomes. Dependent t-tests and repeated measures ANOVA will be used to evaluate within sub-groups variability. Statistical analysis is performed using GraphPad PRISM 7.

### Outcomes

The primary outcome for this study is the analysis of severity of symptoms experienced by the individuals over the 7 days of taking the NAM/PT composition. Components of symptom severity include development of symptoms and severity of each symptom, such as hot flashes, hot sweats at night, bloating, and difficulty sleeping.

Secondary outcomes for this study include a more in-depth analysis of primary outcomes, including changes in the levels of estradiol and NAD metabolites measured from urine samples.

It is expected that the combination of NAM and PT results in a reduction of the subject's menopause symptoms, including reducing the severity or frequency one or more of the following symptoms: hot flashes, poor sleep (decreased sleep quality), bloating, mood swings, irritability, dry skin, and vaginal dryness.

### Example 4: Reducing the Severity and/or Frequency of Menopausal Symptoms Using Nicotinic Acid and Pterostilbene

A single arm study is performed to determine whether supplementation with a composition containing nicotinic acid (NA) and pterostilbene (PT) increases the natural production of estradiol, measured in urinary waste. The secondary objective is to determine whether through increased estradiol levels, the undesirable menopausal effects, assessed via questionnaires, are mitigated by a short-term supplementation with the NA/PT composition. The composition is an encapsulated mixture of NA and PT.

Up to 40 healthy women over 35 years of age experiencing undesirable menopause-related discomfort are entered in this study. In a study questionnaire to be given on day 0 and day 8, subjects will be asked to report discomfort includes mood-swings, hot flashes, poor sleep, bloating, and dry skin. Potential subjects are excluded if they are under 35 year of age, pregnant or lactating, actively participating in another clinical trial, a member of a prison system, currently receiving hormone replacement therapy, currently undergoing active cancer treatment, or currently using an immunosuppressant.

Following completion of the consent form, the subjects are asked to fill a pre-supplementation questionnaire. They then are asked to provide a urine sample after being given a urine sampling kit. The questionnaire seeks information on their current state in view of symptoms associated with menopausal discomfort in addition to demographics (age, sex, race, ethnicity). The subject is also supplied with their first supply of the NA/PT composition (day 0) (small tub containing 7 days of capsules). The subject will take 2 capsules once a day (total daily dose contains 125 mg of NA and 50 mg of PT). At day 8, the subjects are asked to provide a second urine in the urine sampling kit provided to them and fill in the same questionnaire administered at enrollment. The questionnaire aims to evaluate the severity and frequency of the most common menopause associated undesirable symptoms.

Once collected and de-identified, urine samples are stored in a dedicated −80° C. freezer until all samples are ready to be processed and analyzed. The urine samples (10 mL) are analyzed for total estradiol content (estrone, estradiol, estriol, and their sulfate and glucoronate metabolites) and for total vitamin B3 metabolites' content.

### Data Analysis

Urine samples undergo a targeted analysis of the NAD and estradiol metabolomic profile, using ultra high-performance liquid chromatography mass spectrometry.

Survey responses are analyzed to determine demographics, and severity of menopausal symptoms.

### Statistical Analysis

This is a short-term metabolic study for which pharmacokinetics and pharmacodynamics outcomes are anticipated. There will be no intention-to-treat methodology in the analysis. There will be no sample stratification. The urine content of the NAD and estradiol metabolomes' measurements will be presented as mean of biological replicates and 95% confidence interval (CI) from all analyzed LC-MS measurements pooled from 2 technical replicates. For multiple comparisons, ANOVA followed by post hoc test, i.e., Bonferroni correction for LC-MS and Dunnett's or Tukey's for combination with questionnaire outcomes. Dependent t-tests and repeated measures ANOVA will be used to evaluate within sub-groups variability. Statistical analysis is performed using GraphPad PRISM 7.

### Outcomes

The primary outcome for this study is the analysis of severity of symptoms experienced by the individuals over the 7 days of taking the NA/PT composition. Components of symptom severity include development of symptoms and severity of each symptom, such as hot flashes, hot sweats at night, bloating, and difficulty sleeping.

Secondary outcomes for this study include a more in-depth analysis of primary outcomes, including changes in the levels of estradiol and NAD metabolites measured from urine samples.

It is expected that the combination of NA and PT results in a reduction of the subject's menopause symptoms, including reducing the severity or frequency one or more of the following symptoms: hot flashes, poor (decreased sleep quality), bloating, mood swings, irritability, dry skin, and vaginal dryness.

### Example 5: Reducing the Severity and/or Frequency of Menopausal Symptoms Using the Reduced Form of Nicotinamide Riboside and Pterostilbene

A single arm study is performed to determine whether supplementation with a composition containing the reduced form of nicotinamide riboside (NRH) and pterostilbene (PT) increases the natural production of estradiol, measured in urinary waste. The secondary objective is to determine whether through increased estradiol levels, the undesirable menopausal effects, assessed via questionnaires, are mitigated by a short-term supplementation with the NRH/PT composition. The composition is an encapsulated mixture of NRH and pterostilbene, BASIS.

Up to 40 healthy women over 35 years of age experiencing undesirable menopause-related discomfort are entered in this study. In a study questionnaire to be given on day 0 and day 8, subjects will be asked to report discomfort includes mood-swings, hot flashes, poor sleep, bloating, and dry skin. Potential subjects are excluded if they are under 35 year of age, pregnant or lactating, actively participating in another clinical trial, a member of a prison system, currently receiving hormone replacement therapy, currently undergoing active cancer treatment, or currently using an immunosuppressant.

Following completion of the consent form, the subjects are asked to fill a pre-supplementation questionnaire. They then are asked to provide a urine sample after being given a urine sampling kit. The questionnaire seeks information on their current state in view of symptoms associated with menopausal discomfort in addition to demographics (age, sex, race, ethnicity). The subject is also supplied with their first supply of the NRH/PT composition (day 0) (small tub containing 7 days of BASIS capsules). The subject will take 2 capsules once a day (total daily dose contains 250 mg of NRH and 50 mg of pterostilbene). At day 8, the subjects are asked to provide a second urine in the urine sampling kit provided to them and fill in the same questionnaire administered at enrollment. The questionnaire aims to evaluate the severity and frequency of the most common menopause associated undesirable symptoms.

Once collected and de-identified, urine samples are stored in a dedicated −80° C. freezer until all samples are ready to be processed and analyzed. The urine samples (10 mL) are analyzed for total estradiol content (estrone, estradiol, estriol, and their sulfate and glucoronate metabolites) and for total vitamin B3 metabolites' content.

### Data Analysis

Urine samples undergo a targeted analysis of the NAD and estradiol metabolomic profile, using ultra high-performance liquid chromatography mass spectrometry.

Survey responses are analyzed to determine demographics, and severity of menopausal symptoms.

### Statistical Analysis

This is a short-term metabolic study for which pharmacokinetics and pharmacodynamics outcomes are anticipated. There will be no intention-to-treat methodology in the analysis. There will be no sample stratification. The urine content of the NAD and estradiol metabolomes' measurements will be presented as mean of biological replicates and 95% confidence interval (CI) from all analyzed LC-MS measurements pooled from 2 technical replicates. For multiple comparisons, ANOVA followed by post hoc test, i.e., Bonferroni correction for LC-MS and Dunnett's or Tukey's for combination with questionnaire outcomes. Dependent t-tests and repeated measures ANOVA will be used to evaluate within sub-groups variability. Statistical analysis is performed using GraphPad PRISM 7.

### Outcomes

The primary outcome for this study is the analysis of severity of symptoms experienced by the individuals over the 7 days of taking the NRH/PT composition. Components of symptom severity include development of symptoms and severity of each symptom, such as hot flashes, hot sweats at night, bloating, and difficulty sleeping.

Secondary outcomes for this study include a more in-depth analysis of primary outcomes, including changes in the levels of estradiol and NAD metabolites measured from urine samples.

It is expected that the combination of NRH and PT results in a reduction of the subject's menopause symptoms, including reducing the severity or frequency one or more of the following symptoms: hot flashes, poor sleep (decreased sleep quality), bloating, mood swings, irritability, dry skin, and vaginal dryness.

### Example 6: Measuring Estradiol Levels Following Treatment with Nicotinamide Riboside and Pterostilbene

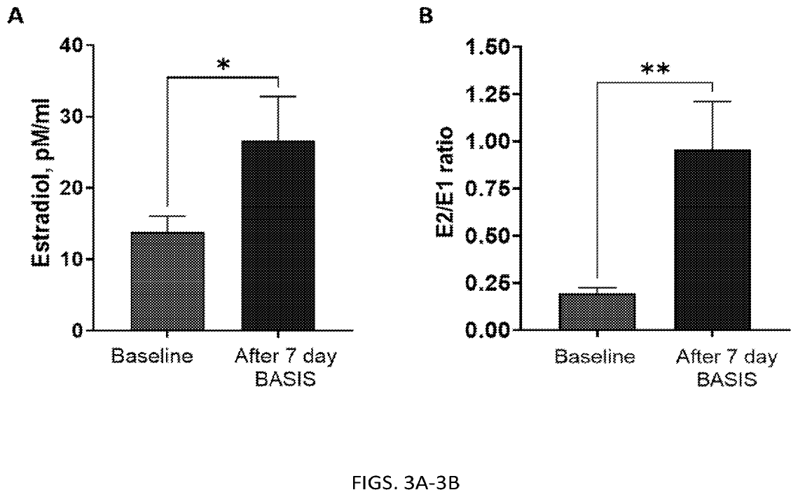

Estradiol levels in urine were measured before and after 7 day supplementation with BASIS. The subjects took 2 capsules once a day (total daily dose contains 250 mg of nicotinamide riboside and 50 mg of pterostilbene). The results are shown in FIG. 3A. Estradiol/Estrone (E2/E1) ratios were calculated from concentrations of estradiol and estrone measured in urine samples collected before and after 7 day BASIS supplementation. The results are shown in FIG. 3B.

Hormone levels were measured by ELISA and levels were normalized to creatinine levels in each sample. Subjects with baseline E2/E1 ratios>1.5 were excluded as these are atypical of perimenopausal or postmenopausal women. Data shown as Mean+SEM. *=P<0.05; **=P<0.01, N=36 subjects. Wilcoxon Pairwise T-test was used for statistical analysis. In this open labeled trial conducted in women with menopausal symptoms, 7 day BASIS supplementation significantly increased estradiol (P<0.05 vs Baseline) and ratio of Estradiol/Estrone (E2/E1, P<0.01 vs Baseline) from baseline. The estradiol level was approximately 60% higher than baseline following treatment with BASIS for 7 days.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of treating a symptom of menopause, comprising administering to a female a composition comprising (a) a therapeutically effective amount of a compound selected from the group consisting of NR, NMN, NAM, NA, NRH, and a mixture thereof, and (b) a therapeutically effective amount of pterostilbene, wherein the female has one or more symptoms of menopause selected from the group consisting of hot flashes, decreased sleep quality, bloating, mood swings, irritability, dry skin, and vaginal dryness.

2. The method of claim 1, wherein the female suffers from vasomotor symptoms of menopause.

3. The method of claim 1 or claim 2, wherein the female suffers from decreased sleep quality.

4. The method of any one of claims 1-3, wherein the composition is administered to the female once per day for at least 7 days.

5. The method of any one of claims 1-4, wherein the female's estradiol level is higher than baseline for at least 2 consecutive days during the treatment period.

6. The method of claim 5, wherein the female's estradiol level is at least 30% higher than baseline following the treatment period.

7. The method of claim 4, wherein an increase in the female's estradiol level from baseline occurs on or by day 7 of the administration of the composition.

8. The method of any one of claims 4-7, wherein there is a decrease in severity of at least one menopausal symptom in the female on or by day 7 of the administration of the composition.

9. The method of any one of claims 4-7, wherein there is a decrease in frequency of at least one menopausal symptom in the female on or by day 7 of the administration of the composition.

10. The method of any one of claims 1-9, wherein the composition is administered parenterally, enterally, intranasally, or transdermally.

11. The method of any one of claims 1-9, wherein the composition is administered orally.

12. The method of any one of claims 1-11, wherein the amount of the compound administered per day is about 10 mg to about 700 mg and the amount of pterostilbene administered per day is about 25 mg to about 200 mg.

13. The method of any one of claims 1-11, wherein the amount of the compound administered per day is about 50 mg to about 250 mg and the amount of pterostilbene administered per day is about 50 mg.

14. A method of treating a symptom of menopause, comprising orally administering to a female for at least 7 days a composition comprising NR and pterostilbene, wherein the amount of NR administered per day is about 200 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

15. The method of claim 14, wherein the amount of NR administered per day is about 250 mg and the amount of pterostilbene administered per day is about 50 mg.

16. The method of claim 14 or 15, wherein the female's estradiol level is higher than baseline for at least 2 consecutive days during the treatment period.

17. The method of claim 16, wherein the female's estradiol level is at least 30% higher than baseline following the treatment period.

18. A method of treating a symptom of menopause, comprising orally administering to a female for at least 7 days a composition comprising NMN and pterostilbene, wherein the amount of NMN administered per day is about 200 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

19. The method of claim 18, wherein the amount of NMN administered per day is about 200 to about 250 mg and the amount of pterostilbene administered per day is about 50 mg.

20. The method of claim 18 or 19, wherein the female's estradiol level is higher than baseline for at least 2 consecutive days during the treatment period.

21. The method of claim 20, wherein the female's estradiol level is at least 30% higher than baseline following the treatment period.

22. A method of treating a symptom of menopause, comprising orally administering to a female for at least 7 days a composition comprising NAM and pterostilbene, wherein the amount of NAM administered per day is about 10 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

23. The method of claim 22, wherein the amount of NAM administered per day is about 20 mg to about 200 mg and the amount of pterostilbene administered per day is about 50 mg.

24. The method of claim 22 or 23, wherein the female's estradiol level is at least 30% higher than baseline following the treatment period.

25. The method of claim 24, wherein the female's estradiol level is at least 50% higher than baseline following the treatment period.

26. A method of treating a symptom of menopause, comprising orally administering to a female for at least 7 days a composition comprising NA and pterostilbene, wherein the amount of NA administered per day is about 10 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

27. The method of claim 26, wherein the amount of NA administered per day is about 20 mg to about 200 mg and the amount of pterostilbene administered per day is about 50 mg.

28. The method of claim 26 or 27, wherein the female's estradiol level is at least 30% higher than baseline following the treatment period.

29. The method of claim 28, wherein the female's estradiol level is at least 50% higher than baseline following the treatment period.

30. A method of treating a symptom of menopause, comprising orally administering to a female for at least 7 days a composition comprising NRH and pterostilbene, wherein the amount of NRH administered per day is about 200 mg to about 700 mg and the amount of pterostilbene administered per day is about 25 mg to about 200 mg, wherein the female has one or more symptoms of menopause selected from the group consisting of hot flashes, decreased sleep quality, bloating, mood swings, irritability, dry skin, and vaginal dryness, and wherein there is a decrease in severity or frequency of at least one menopausal symptom in the female on or by day 7 of the administration of the composition.

31. The method of claim 30, wherein the amount of NRH administered per day is about 200 mg to about 250 mg and the amount of pterostilbene administered per day is about 50 mg.

32. The method of claim 30 or 31, wherein the female's estradiol level is at least 30% higher than baseline following the treatment period.

33. The method of claim 32, wherein the female's estradiol level is at least 50% higher than baseline following the treatment period.
